(19) United States
(12) Patent Application Publication
(10) Pub. No.: US 2011/0118357 A1
(43) Pub. Date: May 19, 2011

(54) PROCESS FOR PREPARING O-DESMETHYLVENLAFAXINE

(75) Inventors: Vinayak Govind Gore, Maharashtra (IN); Vikas Sharad Kulkarni, Maharashtra (IN); Madhukar Shaligram Patil, Maharastra (IN)

(73) Assignees: Generics Limited; Mylan India Private Limited

(21) Appl. No.: 12/739,431

(22) PCT Filed: Oct. 18, 2008
(86) PCT No.: PCT/GB08/50962

§ 371 Date: Nov. 23, 2010

(30) Foreign Application Priority Data
Oct. 26, 2007 (IN) 2122/MUM/2007

(51) Int. Cl. A61K 31/137; C07C 215/42; A61P 25/22; A61P 25/24; A61P 13/00; A61P 25/30; A61P 25/32; A61P 25/18; A61P 15/00

(52) U.S. Cl. 514/649; 564/360

(57) ABSTRACT

The present invention provides a convenient and efficient process for the preparation of O-desmethylvenlafaxine (ODV), comprising the reaction of venlafaxine, or a salt thereof, with a thiol reagent such as a dithiol, an aminothiol or an inorganic thiol. The present invention also provides a process for purifying ODV base, said process comprising the steps of mixing crude ODV base with an alcohol to form a suspension and adding acid followed by base to generate ODV base with high purity.

### CROSS-REFERENCE TO RELATED APPLICATION(s)

This application is a Section 371 National Stage Application of International No. PCT/GB2008/0050962, filed 18 Oct. 2008 and published as WO 2009/053731 A2 on 30 Apr. 20089, which claims priority from the IN Patent Application No. 2122/MUM/2007, filed 26 Oct. 2007, the contents of which are incorporated herein in their entirety for all purposes.

### FIELD OF THE INVENTIONS

The present invention provides a convenient and efficient process for the preparation of O-desmethylvenlafaxine (ODV), comprising the reaction of venlafaxine, or a salt thereof, with a thiol reagent such as a dithiol, an aminothiol or an inorganic thiol. The present invention also provides a process for purifying ODV base, said process comprising the steps of mixing crude ODV base with an alcohol to form a suspension and adding acid followed by base to generate ODV base with high purity.

### BACKGROUND OF THE INVENTION

O-Desmethylvenlafaxine (ODV, II), chemically named 1-[1-(4-hydroxyphenyl)-2-(dimethylamino)-ethyl]-cyclohexanol, is a major metabolite of venlafaxine. ODV is known to inhibit norepinephrine and serotonin uptake and to have antidepressant activity. It has been further reported that oral administration of ODV succinate, in particular in sustained release form, results in a lower incidence of nausea, vomiting, diarrhea, abdominal pain, headache, vaso-vagal malaise and/or trismus than oral administration of venlafaxine. ODV is known to be effective in treating patients suffering from depression, anxiety and panic disorder.

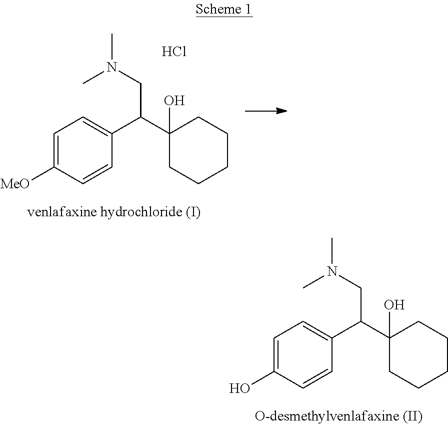

Various prior art patents and patent applications describe processes for the preparation of ODV free base, which can be converted into a desired pharmaceutically acceptable salt. Such prior art processes to obtain ODV are disclosed in documents U.S. Pat. No. 4,535,186, U.S. Pat. No. 6,673,838, U.S. Pat. No. 4,761,501, WO 03/48104, WO 00/59851, WO 00/32556, WO 00/76955, WO 00/32555, WO 02/64543, WO 2007/071404 and U.S. Pat. No. 6,689,912.

The process described in U.S. Pat. No. 4,535,186 for the preparation of ODV leads to relatively low yields and throughput as benzyl protecting groups are used.

Other prior art patents and patent applications listed above describe processes for making ODV which avoid using protecting groups as demethylation of venlafaxine is used instead (Scheme 1). However, in general, the substituted phenoxy group of venlafaxine is a very stable moiety and thus the demethylation reaction typically requires special reagents and drastic conditions. Furthermore, the reagent must be carefully selected so that it does not attack the tertiary hydroxy group on the cyclohexane ring on venlafaxine.

The starting material, venlafaxine, or its salt, may be prepared in accordance with procedures known in the art, such as in patent U.S. Pat. No. 4,535,186.

WO 00/59851, WO 00/32556 and WO 00/32555 disclose a process for preparing ODV starting from venlafaxine using lithium diphenyl phosphide (prepared in-situ from diphenyl phosphine and n-butyl lithium) as the demethylation agent and tetrahydrofuran as a solvent. However, disadvantages of this process are that the concentration of the material in the solvent is very low and the presence of a largely insoluble lithium salt of venlafaxine which is formed in the tetrahydrofuran solvent.

WO 02/64543 discloses a process for the preparation of ODV by demethylation of venlafaxine using reagents such as L-selectride. However, this process is relatively expensive due to the cost of the reagents.

Processes are also disclosed describing demethylation using boron tribromide as the reagent. However, this process suffers from the major disadvantages of the requirement of low temperature and the hazards involved in the use of boron tribromide. Consequently, this process is not amenable on large scale.

WO 02/64543 and WO 03/48104 disclose a demethylation process using the sodium salt of dodecanethiol in polyethylene glycol 400 at 190° C.-200° C. This process suffers from the disadvantage that the decomposition of ODV is unavoidable at such high temperatures. In addition, there is the need to employ two solvents: methanol for formation of the suspension of sodium methoxide and then polyethylene glycol 400 to run the reaction at high temperature. This necessitates removal of methanol from the reaction mixture to attain high temperature and to drive the reaction to completion.

WO 00/76955 discloses a demethylation process using the sodium salt of ethane thiol, however this process suffers from the disadvantage of not being very high yielding and affording a product of low purity. The use of the low boiling ethane thiol (b.p. 35° C.) means that handling and storage of the reagent on an industrial scale is difficult and has safety problems. In addition, ethane thiol is very toxic and has a very noxious smell which is also not suitable for industrial manufacture. In addition, the use of sodium hydride to form the sodium salt of ethane thiol is also not convenient on a commercial scale.

WO 2007/071404 discloses the use of sodium sulfide as the reagent for demethylation of venlafaxine. However, the process has the disadvantage of requiring an inconvenient, prolonged reaction time of around 30 hours.

Thus, the processes disclosed in the prior art suffer from several disadvantages such as moderate to low yields; obtaining ODV (II) in an impure state; very high temperatures; lengthy processes; and/or using expensive, toxic and/or hazardous reagents, which are not recommended to be used on a commercial scale, such as L-Selectride, ethane thiol, boron tribromide and n-butyl lithium.

Therefore, it would be desirable to develop an alternative efficient, non-hazardous and economical process for demethylation of venlafaxine to obtain ODV.

### SUMMARY OF THE INVENTION

The present invention provides a new, efficient, non-hazardous and economical process for converting venlafaxine into ODV by demethylation.

According to the first aspect of the invention there is provided a process for the preparation of O-desmethylvenlafaxine (ODV, II), or a pharmaceutically acceptable salt thereof, comprising the reaction of venlafaxine, or a salt thereof, with a thiol reagent.

The term ‘thiol reagent’ as used herein throughout the description and claims can mean a thiol, oligo- or polythiol such as a dithiol or trithiol and/or their anions and/or salts thereof. The thiol reagent is preferably a dithiol or a salt or anion thereof such as 1,4-benzenedimethanethiol, biphenyl-4,4′-dithiol, 1,4-butanedithiol, 2,3-butanedithiol, 1,2-ethane dithiol, 2,2′-(ethylenedioxy)diethanethiol, 1,16-hexadecanedithiol, 1,6-hexanedithiol, 1,8-octanedithiol, 1,9-nonanedithiol, 1,5-pentanedithiol, 1,3-propanedithiol, 1,2-propanedithiol or 1,11-undecanedithiol. The thiol reagent is preferably a low molecular weight thiol reagent. Most preferably the thiol reagent is a low molecular weight dithiol reagent. Preferably the thiol reagent has a molecular weight in its non-salt form of less than 200 Da, more preferably the thiol reagent has a molecular weight in its non-salt form of less than 150 Da. Preferably the thiol reagent has a molecular weight in its non-salt form of at least 65 Da, or at least 75 Da, or at least 90 Da.

In one embodiment, the thiol reagent is selected from an alkyl, alkenyl, alkynyl, aryl, arylalkyl, arylalkenyl, arylalkynyl, alkylaryl, alkenylaryl or alkynylaryl thiol, each of which may optionally be substituted.

Preferably, the thiol reagent is selected from an optionally substituted alkyl, aryl, arylalkyl or alkylaryl thiol, preferably from an optionally substituted alkyl, arylalkyl or aryl thiol, such as a straight-chained or branched alkyl or arylalkyl thiol reagent. Optionally the thiol reagent is prepared in-situ from an unsubstituted or substituted episulfide having alkyl, aryl, arylalkyl or alkylaryl substituents, preferably having alkyl, aryl or alkylaryl substituents.

Preferably the thiol reagent does not contain an aromatic group. In one embodiment the thiol reagent contains 1 to 20 carbon atoms, preferably the thiol reagent contains 1 to 10 carbon atoms, most preferably the thiol reagent contains 2 to 4 carbon atoms. Preferably the thiol reagent is an aliphatic dithiol containing 1 to 20 carbon atoms such as 1,4-butanedithiol, 2,3-butanedithiol, 1,2-ethane dithiol, 2,2′-(ethylenedioxy)diethanethiol, 1,16-hexadecanedithiol, 1,6-hexanedithiol, 1,8-octanedithiol, 1,9-nonanedithiol, 1,5-pentanedithiol, 1,3-propanedithiol, 1,2-propanedithiol or 1,11-undecanedithiol, and most preferably the aliphatic dithiol is 1,2-ethane dithiol.

For the purposes of the present invention, an ‘alkyl’ group is defined as a monovalent saturated hydrocarbon, which may be straight-chained or branched, or be or include cyclic groups. An alkyl group may optionally include one or more heteroatoms N, O or S in its carbon skeleton. Examples of alkyl groups are methyl, ethyl, n-propyl, i-propyl, n-butyl, i-butyl, t-butyl and n-pentyl groups. Preferably an alkyl group is straight-chained or branched and does not include any heteroatoms in its carbon skeleton. Preferably an alkyl group is a C₁-C₁₂ alkyl group, which is defined as an alkyl group containing from 1 to 12 carbon atoms. More preferably an alkyl group is a C₁-C₆ alkyl group, which is defined as an alkyl group containing from 1 to 6 carbon atoms. An ‘alkylene’ group is similarly defined as a divalent alkyl group.

An ‘alkenyl’ group is defined as a monovalent hydrocarbon, which comprises at least one carbon-carbon double bond, which may be straight-chained or branched, or be or include cyclic groups. An alkenyl group may optionally include one or more heteroatoms N, O or S in its carbon skeleton. Examples of alkenyl groups are vinyl, allyl, but-1-enyl and but-2-enyl groups.

Preferably an alkenyl group is straight-chained or branched and does not include any heteroatoms in its carbon skeleton. Preferably an alkenyl group is a C₂-C₁₂ alkenyl group, which is defined as an alkenyl group containing from 2 to 12 carbon atoms. More preferably an alkenyl group is a C₂-C₆ alkenyl group, which is defined as an alkenyl group containing from 2 to 6 carbon atoms. An ‘alkenylene’ group is similarly defined as a divalent alkenyl group.

An ‘alkynyl’ group is defined as a monovalent hydrocarbon, which comprises at least one carbon-carbon triple bond, which may be straight-chained or branched, or be or include cyclic groups. An alkynyl group may optionally include one or more heteroatoms N, O or S in its carbon skeleton. Examples of alkynyl groups are ethynyl, propargyl, but-1-ynyl and but-2-ynyl groups. Preferably an alkynyl group is straight-chained or branched and does not include any heteroatoms in its carbon skeleton. Preferably an alkynyl group is a C₂-C₁₂ alkynyl group, which is defined as an alkynyl group containing from 2 to 12 carbon atoms. More preferably an alkynyl group is a C₂-C₆ alkynyl group, which is defined as an alkynyl group containing from 2 to 6 carbon atoms. An ‘alkynylene’ group is similarly defined as a divalent alkynyl group.

An ‘aryl’ group is defined as a monovalent aromatic hydrocarbon. An aryl group may optionally include one or more heteroatoms N, O or S in its carbon skeleton. Examples of aryl groups are phenyl, naphthyl, anthracenyl and phenanthrenyl groups. Preferably an aryl group does not include any heteroatoms in its carbon skeleton. Preferably an aryl group is a C₄-C₁₄ aryl group, which is defined as an aryl group containing from 4 to 14 carbon atoms. More preferably an aryl group is a C₆-C₁₀ aryl group, which is defined as an aryl group containing from 6 to 10 carbon atoms. An ‘arylene’ group is similarly defined as a divalent aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

For the purposes of this invention, an optionally substituted hydrocarbon or alkyl, alkenyl, alkynyl, aryl, arylalkyl, arylalkenyl, arylalkynyl, alkylaryl, alkenylaryl or alkynylaryl group, such as for example an optionally substituted alkyl thiol, may be substituted with one or more of —F, —Cl, —Br, —I, —CF₃, —CCl₃, —CBr₃, —Cl₃, —OH, —SH, —NH₂, —CN, —NO₂, —COOH, —R^{α}—O—R^{β}, —R^{α}—S—R^{β}, —R^{α}—SO—R^{β}, —R^{α}-SO₂-R^{β}, —R^{α}—SO₂—OR^{β}, —R^{αO—SO}₂—R^{β}, —R —SO₂—N(R^{β})₂, —R^{α}—NR^{β}—SO₂—R^{β}, —R^{α}O—SO₂—OR^{β}, —R^{αO—SO}₂—N(R^{β})₂, —R^{α}—NR^{β}—SO₂—OR^{β}, —R^{α}—NR^{β}—SO₂—N(R^{β})₂, —R^{α}—N(R^{β})₂, —R^{α}—N(R^{β})₃⁺, —R^{α}—P(R^{β})₂, —R^{α}—Si(R^{β})₃, —R^{α}—CO—R^{β}, —R^{α}—CO—OR^{β}, —R^{αO—CO—R}^{β}, —R^{α}—CO—N(R^{β})₂, —R^{α}—NR^{β}—CO—R^{β}, R^{α}O—CO—OR^{β}, —R^{α}O—CO—N(R^{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{αO—CS—R}^{β}, —R CS—N(R^{β})₂, —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}, a bridging substituent such as —O—, —S—, —NR^{β}— or —R^{α}—, or a π-bonded substituent such as ═O, ═S or ═NR^{β}. In this context, —R^{α}— is independently a chemical bond, a C₁-C₁₀ alkylene, C₁-C₁₀ alkenylene or C₁-C₁₀ alkynylene group. —R^{β} is independently hydrogen, unsubstituted C₁-C₆ alkyl or unsubstituted C₆-C₁₀ aryl. Optional substituent(s) are taken into account when calculating the total number of carbon atoms in the parent group substituted with the optional substituent(s). Preferably an optionally substituted hydrocarbon or alkyl, alkenyl, alkynyl, aryl, arylalkyl, arylalkenyl, arylalkynyl, alkylaryl, alkenylaryl or alkynylaryl group is not substituted with a bridging substituent. Preferably an optionally substituted hydrocarbon or alkyl, alkenyl, alkynyl, aryl, arylalkyl, arylalkenyl, arylalkynyl, alkylaryl, alkenylaryl or alkynylaryl group is not substituted with a π-bonded substituent. Preferably a substituted group comprises 1, 2 or 3 substituents, more preferably 1 or 2 substituents, and even more preferably 1 substituent.

Any optional substituent may be protected. Suitable protecting groups for protecting optional substituents are known in the art, for example from ‘Protective Groups in Organic Synthesis’ by T. W. Greene and P. G. M. Wuts (Wiley-Interscience, 4^{th} edition, 2006).

Preferably, the thiol reagent is an aminothiolate anion or an aminothiol, optionally having 1 to 20 carbon atoms, preferably having 1 to 10 carbon atoms, more preferably having 2 to 4 carbon atoms. Preferably the amino group of the aminothiol or aminothiolate anion is unsubstituted or substituted with one or more optionally substituted alkyl, alkenyl, alkynyl, aryl, arylalkyl, arylalkenyl, arylalkynyl, alkylaryl, alkenylaryl or alkynylaryl groups or mixtures thereof. More preferably the amino group is unsubstituted or substituted with one or more alkyl, aryl or arylalkyl groups or mixtures thereof. Preferably the aminothiolate anion or aminothiol is an N,N-dialkylaminoalkane thiol and most preferably, 2-diethylaminoethane thiol, Et₂N-CH₂CH₂—SH.

As used herein, an ‘aminothiol’ refers to a hydrocarbon (i.e. an optionally substituted compound comprising carbon and hydrogen) substituted with both an amino group and a thiol group (i.e. —SH). An ‘aminothiolate anion’ is similarly defined as a hydrocarbon substituted with both an amino group and an anionic thiolate group (i.e. —S⁻).

The thiol reagent can also be an inorganic thiol, i.e. a reagent of formula M⁺SH⁻, wherein M⁺ is any cationic species, such as sodium thiol. Preferably M is a metal, more preferably M is an alkaline metal such as Li, Na or K.

The reaction solvent is preferably selected from an alcohol, ethylene glycol, an ether of ethylene glycol or a mixture thereof, such as polyethylene glycol (e.g. polyethylene glycol 400), cellosolve (such as 2-ethoxy-ethanol and 2-methoxy-ethanol) or 1-butanol.

Preferably the reaction solvent is a single solvent. Preferably the reaction solvent has a boiling point of at least 100° C., more preferably at least 115° C., more preferably at least 130° C.

In preferred embodiments of the current invention, a thiolate anion is generated by treatment of the thiol reagent with a base, such as an alkoxide in the reaction solvent. Preferably the alkoxide is not generated in-situ. Preferably the alkoxide is added to the reaction solvent as a solid in the form of a metal salt. The alkoxide is preferably a t-butoxide, most preferably potassium t-butoxide.

Preferably, the process according to the invention is performed at a temperature within the range of 100° C. to 220° C., more preferably within the range of 120° C. to 150° C., and most preferably within the range of 130° C. to 135° C. This temperature range is particularly preferred when the thiol reagent is an oligo- or polythiol or a salt or an anion thereof such as a dithiol or a salt or anion thereof.

In one embodiment, wherein the thiol reagent is an aminothiol or an aminothiolate ion, the process according to the invention is preferably performed at a temperature within the range of 150° C. to 190° C., more preferably within the range of 170° C. to 175° C.

In another embodiment, wherein the thiol reagent is an inorganic thiol, the process according to the invention is preferably performed at a temperature within the range of 130° C. to 170° C., more preferably within the range of 150° C. to 155° C.

Preferably the venlafaxine or salt thereof is allowed to react with the thiol reagent for between 6 and 36 hours, more preferably for between 12 and 30 hours, most preferably for between 24 and 28 hours.

Preferably, during the work up procedure of the current invention, to remove process impurities, the product is washed with a hydrocarbon solvent, such as cyclohexane, toluene, xylene or mixtures thereof, or a halogenated hydrocarbon solvent, such as dichloromethane, ethylene dichloride or mixtures thereof Preferably the product is washed with an aromatic hydrocarbon solvent or a halogenated hydrocarbon solvent.

Preferably, a pharmaceutically acceptable salt of ODV prepared by the current invention is selected from the succinate or fumarate salt. A salt of venlafaxine used in the current invention is preferably the hydrochloride salt.

Preferably, the crude ODV base formed by the process of the current invention is purified by mixing with an alcohol, such as methanol, ethanol or isopropanol or a mixture thereof, to form a suspension and then adding acid followed by base to generate ODV base with high purity.

Preferably sufficient acid is added to dissolve all or substantially all of the solid crude ODV base in the suspension, and the ODV base with high purity is formed by precipitation on addition of the base.

In a second aspect of the present invention there is provided a process for purifying ODV base, said process comprising the steps of mixing crude ODV base with an alcohol, such as methanol, ethanol or isopropanol or a mixture thereof, to form a suspension and adding acid followed by base to generate ODV base with high purity. Preferably sufficient acid is added to dissolve all or substantially all of the solid crude ODV base in the suspension, and the ODV base with high purity is formed by precipitation on addition of the base.

In the first and second aspects of the present invention, most preferably the alcohol used in the purification of crude ODV base is methanol. Preferably, the acid used is an inorganic acid, such as hydrochloric acid or sulfuric acid, and the base used is an organic base such as triethylamine or trimethylamine. Alternatively, the base used can be an inorganic base, such as ammonia, sodium carbonate, potassium carbonate or sodium hydroxide. Preferably the ODV base generated is at least 95% pure, at least 98% pure, at least 99% pure, at least 99.5% pure, or at least 99.9% pure. Most preferably the ODV base generated is at least 99.99% pure. Preferably the purity is as analysed by HPLC.

The process according to the first or second aspects of the invention is preferably carried out such that the ODV or pharmaceutically acceptable salt thereof is obtained in a yield of 25% or more, preferably 30% or more, preferably 50% or more, preferably 60% or more, preferably 70% or more, preferably 80% or more, preferably 85% or more.

Preferably, wherein the thiol reagent is an oligo- or polythiol or a salt or an anion thereof such as a dithiol or a salt or anion thereof, the process is carried out such that the ODV or pharmaceutically acceptable salt thereof is obtained in a yield of 50% or more, preferably 60% or more, preferably 70% or more, preferably 80% or more, preferably 85% or more.

Preferably, wherein the thiol reagent is an aminothiol or an aminothiolate ion, the process is carried out such that the ODV or pharmaceutically acceptable salt thereof is obtained in a yield of 30% or more, preferably 50% or more, preferably 60% or more, preferably 70% or more, preferably 80% or more, preferably 85% or more.

Preferably the process according to the first or second aspects of the invention is carried out on an industrial scale, preferably to obtain batches of ODV or a pharmaceutically acceptable salt thereof of 100 g, 500 g, 1 kg, 5 kg, 10 kg, 50 kg, 100 kg or more.

In a third aspect of the current invention, there is provided ODV or a pharmaceutically acceptable salt thereof prepared by a process according to the first or second aspects of the invention. Preferred salts of the third aspect of the invention are the succinate and fumarate salts. Preferably the ODV or pharmaceutically acceptable salt thereof is suitable for use in medicine, preferably for treating or preventing depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.

In a fourth aspect of the current invention, there is provided a pharmaceutical composition comprising ODV or a pharmaceutically acceptable salt thereof prepared by a process according to the first or second aspects of the invention. Preferably, the pharmaceutical composition according to the fourth aspect of the invention comprises ODV succinate or ODV fumarate.

In a fifth aspect of the current invention, there is provided the use of a pharmaceutical composition according to the fourth aspect of the invention for the preparation of a medicament for the treatment or prevention of depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.

In a sixth aspect of the current invention, there is provided a method of treating or preventing depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease, the method comprising administering to a patient in need thereof a therapeutically or prophylactically effective amount of ODV or a pharmaceutically acceptable salt thereof according to the third aspect of the invention. Preferably the patient is a mammal, preferably a human.

### DETAILED DESCRIPTION OF THE INVENTION

ODV base and its salts exist as enantiomers and the present invention includes racemic mixtures as well as stereoisomerically pure forms of the same. The term ‘ODV’ as used herein refers to the racemic mixtures and stereoisomerically pure forms of ODV, unless otherwise indicated. The term ‘stereoisomerically pure’ refers to compounds, which are comprised of a greater proportion of the desired isomer than that of the optical antipode. A stereoisomerically pure ODV free base is generally made up of at least 90% of the desired isomer based upon 100% total weight of ODV free base.

One advantage of the present invention is use of a commercially readily available thiol reagent, such as 1,2-ethane dithiol, which is safe and convenient to handle on a commercial scale. The use of this type of thiol reagent has significant impact on the scaling up of the process to provide commercial sized batches and, in addition, there are improvements in yield and purity over prior art processes.

As discussed above, the present invention provides a novel process for the preparation of highly pure ODV free base which can be easily adopted for commercial production with a high degree of consistency in quality and yield. The ODV base prepared by the current invention can be subsequently converted into pharmaceutically acceptable salts, such as the succinate or fumarate salts, for finished dosage form preparation.

A further advantage of the present invention is the improved process for preparation of the thiolate anion in the same reaction solvent that is used to perform the demethylation. This offers a significant advantage by way of using one solvent for the whole sequence. Conversely, in the process described in U.S. Pat. No. 6,689,912 for demethylation of venlafaxine, the sodium salt of dodecanethiolate has been prepared in methanol followed by further treatment with venlafaxine in polyethylene glycol 400. To drive the reaction to completion, methanol needs to be distilled off. This cumbersome procedure is avoided with the present invention.

Moreover, the present invention provides a process for the preparation of ODV base wherein the reaction can be performed at a temperature between 130° C. to 135° C. which can be easily achieved on commercial scale and affords less impurities in the finished product.

Yet another advantage of preferred aspects of the present invention is the improved process for the preparation, isolation and purification of ODV base in high yield, approximately 85% molar yield, with high purity conforming to ICH guidelines of impurity profile. The processes of the current invention are capable of providing ODV base with consistent chemical purity irrespective of the scale of preparation.

In addition, the current invention offers a simple work up procedure with improved yield and quality with minimum contamination with process impurities. Therefore the process of the current invention is amenable to large-scale production wherein reaction conditions can be easily controlled. Additionally, the product obtained by following the process disclosed here is readily filtered and easily dried.

The present invention further provides a process for the preparation of ODV base by reacting an anion of either a dithiol, an aminothiol or anhydrous sodium thiol with venlafaxine base or a salt of venlafaxine in a suitable solvent at a much lower temperature than that reported in the prior art for similar methods.

A further advantage of the current invention is that the demethylation reaction can be performed on venlafaxine hydrochloride as well as venlafaxine free base.

Preferably, the process of the current invention is performed in the presence of a protic or aprotic solvent and, optionally, a base such as an alkoxide is used to generate the thiolate anion. As used herein, the term ‘alkoxide’ means alkyl-O⁻. The alkoxide is preferably comprised of a straight-chained or branched alkyl group of 1 to 6 carbon atoms, which may be substituted or unsubstituted, and is a salt of a metal, such as lithium, sodium, potassium, calcium, magnesium or a salt of ammonia or an alkylammonia. The alkoxide base is most preferably potassium t-butoxide or sodium methoxide, preferably potassium t-butoxide. Preferably the thiolate anion is prepared in-situ in the same solvent used for running the reaction.

The solvent used in the reaction mixture is preferably an alcoholic or ethereal solvent, more preferably an alcohol such as 1-butanol, methyl cellosolve, ethyl cellosolve or polyethylene glycol. Preferably the solvent is inert, polar and high boiling, most preferably the solvent is polyethylene glycol 400.

Preferably, purification of crude ODV base is carried out by forming a suspension of crude ODV in methanol and adding aqueous hydrochloric acid followed by aqueous ammonia to obtain ODV base with high purity conforming to ICH guidelines.

In a preferred embodiment of the current invention there is provided a process for the preparation of ODV base or its pharmaceutically acceptable salts comprising:

(a) reacting a thiol reagent with an appropriate alkoxide to form a thiolate or dithiolate anion (preferably a dithiolate anion) in polyethylene glycol 400;
(b) reacting the thiolate or dithiolate anion with venlafaxine free base or a salt of venlafaxine such as venlafaxine hydrochloride in polyethylene glycol 400 at a temperature in the range of 130° C.-135° C.;
(c) isolating the crude ODV base at pH>9.5;
(d) washing the crude ODV base with dichloromethane to remove impurities; and
(e) forming a suspension of crude ODV in methanol and adding aqueous hydrochloric acid followed by aqueous ammonia to obtain pure ODV base.

In a preferred embodiment of the current invention there is provided a process for the preparation of ODV base or its pharmaceutically acceptable salts comprising:

(a) reacting a thiol reagent with an appropriate alkoxide to form a thiolate or dithiolate anion (preferably a dithiolate anion) in polyethylene glycol 400;
(b) reacting the thiolate or dithiolate anion with venlafaxine free base or a salt of venlafaxine such as venlafaxine hydrochloride in polyethylene glycol 400 at a temperature in the range of 130° C.-135° C.;
(c) quenching the reaction mixture with water;
(d) washing the reaction mixture with dichloromethane to remove impurities;
(e) isolating the crude ODV base at pH>9.5; and
(f) forming a suspension of crude ODV in methanol and adding aqueous hydrochloric acid followed by aqueous ammonia to obtain pure ODV base.

The thiol reagent used in either of the above two preferred embodiments is most preferably 1,2-ethane dithiol and the alkoxide is preferably potassium t-butoxide. The use of this combination of reagents is very safe and efficient on a commercial scale. 1,2-Ethane dithiol is much less toxic and noxious than other thiols such as ethane thiol. Surprisingly this combination of reagents also affords a very pure product in high yield.

In an alternative preferred embodiment of the current invention there is provided a process for the preparation of ODV base or its pharmaceutically acceptable salts comprising:

(a) reacting an aminothiol as its hydrochloride with an appropriate alkoxide to form an aminothiolate anion in polyethylene glycol 400;
(b) reacting the aminothiolate anion with venlafaxine free base or a salt of venlafaxine such as venlafaxine hydrochloride in polyethylene glycol 400 at a temperature in the range of 150° C.-155° C.;
(c) isolating the crude ODV base at pH>9.5;
(d) washing the crude ODV base with dichloromethane to remove impurities; and
(e) forming a suspension of crude ODV in methanol and adding aqueous hydrochloric acid followed by aqueous ammonia to obtain pure ODV base.

In another alternative preferred embodiment of the current invention there is provided a process for the preparation of ODV base or its pharmaceutically acceptable salts comprising:

(a) reacting an aminothiol as its hydrochloride with an appropriate alkoxide to form an aminothiolate anion in polyethylene glycol 400;
(b) reacting the aminothiolate anion with venlafaxine free base or a salt of venlafaxine such as venlafaxine hydrochloride in polyethylene glycol 400 at a temperature in the range of 170° C.-175° C.;
(c) quenching the reaction mixture with water;
(d) washing the reaction mixture with dichloromethane to remove impurities;
(e) isolating the crude ODV base at pH>9.5; and
(f) forming a suspension of crude ODV in methanol and adding aqueous hydrochloric acid followed by aqueous ammonia to obtain pure ODV base.

In a further preferred embodiment of the current invention there is provided a process for the preparation of ODV base or its pharmaceutically acceptable salts comprising:

(a) reacting anhydrous sodium thiol with venlafaxine free base or a salt of venlafaxine such as venlafaxine hydrochloride in polyethylene glycol 400 at a temperature in the range of 190° C.-195° C.;
(b) isolating the crude ODV base at pH>9.5;
(c) washing the crude ODV base with dichloromethane to remove impurities; and
(d) forming a suspension of crude ODV in methanol and adding aqueous hydrochloric acid followed by aqueous ammonia to obtain pure ODV base.

In yet a further preferred embodiment of the current invention there is provided a process for the preparation of ODV base or its pharmaceutically acceptable salts comprising:

(a) reacting anhydrous sodium thiol with venlafaxine free base or a salt of venlafaxine such as venlafaxine hydrochloride in polyethylene glycol 400 at a temperature in the range of 150° C.-155° C.;
(b) quenching the reaction mixture with water;
(c) washing the reaction mixture with dichloromethane to remove impurities;
(d) isolating the crude ODV base at pH>9.5; and
(e) forming a suspension of crude ODV in methanol and adding aqueous hydrochloric acid followed by aqueous ammonia to obtain pure ODV base.

The present invention further provides a pharmaceutical composition comprising the ODV, or pharmaceutically acceptable salts thereof, which have been prepared in accordance with the first or second aspects of the invention. It also provides for the use of the aforesaid pharmaceutical compositions for the preparation of a medicament for the treatment or prevention of depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.

The dosage form can be a solution or suspension form but is preferably solid and comprises one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a gelatin material and can include a conventionally prepared granulate of excipients and adduct or solvate in accordance with the invention.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

The following paragraphs enumerated consecutively from 1 through 57 provide for various aspects of the present invention. In one embodiment, the present invention provides:

1. A process for the preparation of O-desmethylvenlafaxine (ODV, II), or a pharmaceutically acceptable salt thereof, comprising the reaction of venlafaxine, or a salt thereof, with a thiol reagent.
2. A process according to paragraph 1, wherein the thiol reagent is a dithiol.
3. A process according to paragraph 1 or 2, wherein the thiol reagent is selected from an alkyl, alkenyl, alkynyl, aryl, arylalkyl, arylalkenyl, arylalkynyl, alkylaryl, alkenylaryl or alkynylaryl thiol, each of which may optionally be substituted.
4. A process according to any one of paragraphs 1 to 3, wherein the thiol reagent is selected from an optionally substituted alkyl, aryl, arylalkyl or alkylaryl thiol or a thiol prepared in-situ from an unsubstituted or substituted episulfide having alkyl, aryl, arylalkyl or alkylaryl substituents.
5. A process according to paragraph 4, wherein the thiol reagent is a straight-chained or branched alkyl or arylalkyl thiol reagent.
6. A process according to any one of paragraphs 1 to 5, wherein the thiol reagent does not contain an aromatic group.
7. A process according to any one of paragraphs 1 to 6, wherein the thiol reagent contains 1 to 20 carbon atoms.
8. A process according to any one of paragraphs 1 to 7, wherein the thiol reagent is an aliphatic dithiol containing 1 to 20 carbon atoms.
9. A process according to paragraph 8, wherein the aliphatic dithiol is 1,2-ethane dithiol.
10. A process according to any one of paragraphs 1 to 8, wherein the thiol reagent is an aminothiolate anion or an aminothiol.
11. A process according to paragraph 10, wherein the aminothiol or aminothiolate anion contains 1 to 20 carbon atoms.
12. A process according to paragraph 10 or 11, wherein the amino group of the aminothiol or aminothiolate anion is unsubstituted or substituted with one or more optionally substituted alkyl, alkenyl, alkynyl, aryl, arylalkyl, arylalkenyl, arylalkynyl, alkylaryl, alkenylaryl or alkynylaryl groups or mixtures thereof.
13. A process according to paragraph 12, wherein the amino group of the aminothiol or aminothiolate anion is either unsubstituted or substituted with one or more alkyl, aryl or arylalkyl groups or mixtures thereof, such as a N,N-dialkylaminoalkane thiol.
14. A process according to paragraph 13, wherein the N,N-dialkylaminoalkane thiol is 2-diethylaminoethane thiol.
15. A process according to paragraph 1 or 2, wherein the thiol reagent is an inorganic thiol.
16. A process according to paragraph 15, wherein the inorganic thiol is sodium thiol.
17. A process according to any one of the preceding paragraphs, wherein the reaction solvent is selected from an alcohol, ethylene glycol, an ether of ethylene glycol or a mixture thereof.
18. A process according to paragraph 17, wherein the reaction solvent is selected from polyethylene glycol (e.g. polyethylene glycol 400), cellosolve or 1-butanol.
19. A process according to any one of the preceding paragraphs, wherein the reaction solvent has a boiling point of at least 100° C.
20. A process according to any one of the preceding paragraphs, wherein a thiolate anion is generated by treatment of the thiol reagent with a base, such as an alkoxide, preferably potassium t-butoxide, in the reaction solvent.
21. A process according to paragraph 20, wherein the alkoxide is not generated in-situ.
22. A process according to any one of the preceding paragraphs, wherein the reaction is performed at a temperature within the range of 100° C. to 220° C.
23. A process according to paragraph 22, wherein the temperature is within the range of 120° C. to 150° C.
24. A process according to paragraph 23, wherein the temperature is within the range of 130° C. to 135° C.
25. A process according to any one of the preceding paragraphs, wherein the venlafaxine or salt thereof is allowed to react with the thiol reagent for between 6 and 36 hours.
26. A process according to paragraph 25, wherein the venlafaxine or salt thereof is allowed to react with the thiol reagent for between 24 and 28 hours.
27. A process according to any one of the preceding paragraphs, wherein during the work up procedure, the product is washed with a hydrocarbon solvent or a halogenated hydrocarbon solvent to remove process impurities.
28. A process according to paragraph 27, wherein the hydrocarbon solvent is selected from cyclohexane, toluene, xylene or mixtures thereof
29. A process according to paragraph 27, wherein the halogenated hydrocarbon solvent is selected from dichloromethane, ethylene dichloride or mixtures thereof.
30. A process according to any one of the preceding paragraphs, wherein the pharmaceutically acceptable salt of ODV prepared is selected from the succinate or fumarate salt.
31. A process according to any one of the preceding paragraphs, wherein the salt of venlafaxine used is the hydrochloride salt.
32. A process according to any one of the preceding paragraphs, wherein the crude ODV base formed is purified by mixing with an alcohol to form a suspension and adding acid followed by base.
33. A process for purifying ODV base, said process comprising the steps of mixing crude ODV base with an alcohol to form a suspension and adding acid followed by base.
34. A process according to paragraph 32 or 33, wherein the alcohol is selected from methanol, ethanol or isopropanol or a mixture thereof
35. A process according to paragraph 34, wherein the alcohol is methanol.
36. A process according to any one of paragraphs 32 to 35, wherein the acid used is an inorganic acid.
37. A process according to paragraph 36, wherein the inorganic acid is hydrochloric acid or sulfuric acid.
38. A process according to paragraph 37, wherein the inorganic acid is hydrochloric acid.
39. A process according to any one of paragraphs 32 to 38, wherein the base used is an organic base.
40. A process according to paragraph 39, wherein the organic base is triethylamine or trimethylamine.
41. A process according to any one of paragraphs 32 to 38, wherein the base used is an inorganic base.
42. A process according to paragraph 41, wherein the inorganic base is ammonia, sodium carbonate, potassium carbonate or sodium hydroxide.
43. A process according to any one of the preceding paragraphs, wherein the ODV or pharmaceutically acceptable salt thereof obtained has a purity of 95% or more (as measured by HPLC).
44. A process according to any one of the preceding paragraphs, wherein the ODV or pharmaceutically acceptable salt thereof is obtained in a yield of 25% or more.
45. A process according to any one of the preceding paragraphs, wherein the process is carried out on an industrial scale.
46. ODV or a pharmaceutically acceptable salt thereof prepared by a process according to any one of the preceding paragraphs.
47. ODV succinate prepared by a process according to any one of paragraphs 1 to 45.
48. ODV fumarate prepared by a process according to any one of paragraphs 1 to 45.
49. ODV or a pharmaceutically acceptable salt thereof according to any one of paragraphs 46 to 48, for use in medicine.
50. ODV or a pharmaceutically acceptable salt thereof according to paragraph 49, for treating or preventing depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.
51. A pharmaceutical composition comprising ODV or a pharmaceutically acceptable salt thereof prepared by a process according to any one of paragraphs 1 to 45.
52. A pharmaceutical composition according to paragraph 51, comprising ODV succinate.
53. A pharmaceutical composition according to paragraph 51, comprising ODV fumarate.
54. Use of a pharmaceutical composition according to any one of paragraphs 51 to 53, for the preparation of a medicament for the treatment or prevention of depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.
55. A method of treating or preventing depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease, the method comprising administering to a patient in need thereof a therapeutically or prophylactically effective amount of ODV or a pharmaceutically acceptable salt thereof according to any one of paragraphs 46 to 50.
56. A method according to paragraph 55, wherein the patient is a mammal.
57. A method according to paragraph 56, wherein the mammal is a human.

The details of the invention, its objects and advantages are explained hereunder in greater detail in the following non-limiting examples.

### EXAMPLES

### Example 1

Preparation of ODV base from venlafaxine base using 1,2-ethane dithiol: 1,2-Ethane dithiol (10.17 g, 0.11 mol) was added to a suspension of potassium t-butoxide (30.29 g, 0.27 mol) in polyethylene glycol 400 (125 mL) at 25° C.-30° C. To this stirred suspension, venlafaxine base (25 g, 0.09 mol) was added and the reaction mixture was heated to 130° C.-135° C. for 24-28 hours. After completion of the reaction, the reaction mixture was allowed to cool to 25° C.-30° C. and water (500 mL) was added followed by addition of conc. hydrochloric acid (35-37% w/v, 30 mL). The solution was extracted with toluene (2×50 mL). To the aqueous solution, 25% w/v aqueous ammonia solution (35 mL) was added to adjust the pH of the solution to >9.5. A solid precipitated out and was filtered to afford crude ODV base. The resultant solid was further suspended in methanol (125 mL) and conc. hydrochloric acid (35-37% w/v, 15 mL) was added to the suspension to dissolve the solid, followed by addition of 25% w/v aqueous ammonia (17 mL) to precipitate out the ODV as an off-white solid. The structure of the compound was confirmed on the basis of ¹H-NMR. Wt of the product=20 g; molar yield=84%; purity (HLPC) in excess of 99.5%.

### Example 2

Preparation of ODV base from venlafaxine hydrochloride using 1,2-ethane dithiol: 1,2-Ethane dithiol (10.17 g, 0.11 mol) was added to a suspension of potassium t-butoxide (30.29 g, 0.27 mol) in polyethylene glycol 400 (125 mL) at 25° C.-30° C. To this stirred suspension, venlafaxine hydrochloride (28 g, 0.09 mol) was added and the reaction mixture was heated to 130° C.-135° C. for 24-28 hours. After completion of the reaction, the reaction mixture was allowed to cool to 25° C.-30° C. and water (500 mL) was added followed by addition of conc. hydrochloric acid (35-37% w/v, 20 mL). The solution was extracted with toluene (2×50 mL). To the aqueous solution, 25% w/v aqueous ammonia solution (25 mL) was added to adjust the pH of the solution to >9.5. A solid precipitated out and was filtered to afford crude ODV base. The resultant solid was further suspended in methanol (140 mL) and conc. hydrochloric acid (35-37% w/v, 17 mL) was added to the suspension to dissolve the solid, followed by addition of 25% w/v aqueous ammonia (20 mL) to precipitate out the ODV as an off-white solid. The structure of the compound was confirmed on the basis of ¹H-NMR. Wt of the product=19.8 g; molar yield=84%; purity (HLPC) in excess of 99.5%.

### Example 3

Preparation of ODV base from venlafaxine base using 2-diethylaminoethane thiol: 2-Diethylaminoethane thiol hydrochloride (3.06 g, 0.018 mol) was added to a suspension of sodium methoxide (2.9 g, 0.054 mol) in polyethylene glycol 400 (50 mL) at 25° C.-30° C. To this stirred suspension, venlafaxine base (2.5 g, 0.009 mol) was added and the reaction mixture was heated to 170° C.-175° C. for 24-28 hours. After completion of the reaction, the reaction mixture was allowed to cool to 25° C.-30° C. and water (200 mL) was added followed by addition of conc. hydrochloric acid (35-37% w/v, 5 mL). The solution was extracted with toluene (2×25 mL). To the aqueous solution, 25% w/v aqueous ammonia solution (7 mL) was added to adjust the pH of the solution to >9.5. A solid precipitated and it was filtered to afford crude ODV base. The resultant solid was further suspended in methanol (12.5 mL) and conc. hydrochloric acid (35-37% w/v, 7.5 mL) was added to the suspension to dissolve the solid, followed by addition of 25% w/v aqueous ammonia (10 mL) to precipitate the ODV as an off-white solid. The structure of the compound was confirmed on the basis of ¹H-NMR. Wt of the product=0.875 g; molar yield=37%; purity (HLPC) in excess of 99.5%.

### Example 4

Preparation of ODV base from venlafaxine hydrochloride using 2-diethylaminoethane thiol: 2-Diethylaminoethane thiol hydrochloride (3.06 g, 0.018 mol) was added to a suspension of sodium methoxide (2.9 g, 0.054 mol) in polyethylene glycol 400 (50 mL) at 25° C.-30° C. To this stirred suspension, venlafaxine hydrochloride (2.8 g, 0.009 mol) was added and the reaction mixture was heated to 170° C.-175° C. for 24-28 hours. After completion of the reaction, the reaction mixture was allowed to cool to 25° C.-30° C. and water (200 mL) was added followed by addition of conc. hydrochloric acid (35-37% w/v, 10 mL). The solution was extracted with toluene (2×25 mL). To the aqueous solution, 25% w/v aqueous ammonia solution (10 mL) was added to adjust the pH of the solution to >9.5. A solid precipitated and it was filtered to afford crude ODV base, which was further suspended in methanol (14 mL) and conc. hydrochloric acid (35-37% w/v, 7.2 mL) was added to the suspension to dissolve the solid, followed by addition of 25% w/v aqueous ammonia (10 mL) to precipitate out the ODV as an off-white solid. The structure of the compound was confirmed on the basis of ¹H-NMR. Wt of the product=0.75 g; molar yield=32%; purity (HLPC) in excess of 99.5%.

### Example 5

Preparation of ODV base from venlafaxine base using sodium thiol as a demethylating reagent: Anhydrous sodium thiol (13 g, 0.23 mol) was prepared by azeotropic removal of water from sodium thiol hydrate. To a stirred suspension of anhydrous sodium thiol in polyethylene glycol 400 (50 mL) at 25° C.-30° C., venlafaxine base (10 g, 0.036 mol) was added and the reaction mixture was heated to 150° C.-155° C. for 24-28 hours. After completion of the reaction, the reaction mixture was allowed to cool to 25° C.-30° C. and water (200 mL) was added followed by addition of conc. hydrochloric acid (35-37% w/v, 25 mL). The solution was extracted with toluene (2×50 mL). To the aqueous solution, 25% w/v aqueous ammonia solution (30 mL) was added to adjust the pH of the solution to >9.5. A solid precipitated and it was filtered to afford crude ODV base, which was further suspended in methanol (50 mL). To this suspension, conc. hydrochloric acid (35-37% w/v, 12.5 mL) was added to dissolve the solid, followed by addition of 25% w/v aqueous ammonia (17.5 mL) to precipitate out the ODV as an off-white solid. The structure of the compound was confirmed on the basis of ¹H-NMR. Wt of the product=3 g; w/w yield=32%; purity (HLPC) in excess of 99.5%.

### Example 6

Preparation of ODV base from venlafaxine hydrochloride using sodium thiol as a demethylating reagent:

Anhydrous sodium thiol (13 g, 0.23 mol) was prepared by azeotropic removal of water from sodium thiol hydrate. To a stirred suspension of anhydrous sodium thiol in polyethylene glycol 400 (50 mL) at 25° C.-30° C., venlafaxine hydrochloride (11.32 g, 0.036 mol) was added. The reaction mixture was heated to 150° C.-155° C. for 24-28 hours. After completion of the reaction, the reaction mixture was allowed to cool to 25° C.-30° C. and water (200 mL) was added followed by addition of conc. hydrochloric acid (35-37% w/v, 30 mL). The solution was extracted with toluene (2×50 mL). To the aqueous solution, 25% w/v aqueous ammonia solution (35 mL) was added to adjust the pH of the solution to >9.5. A solid precipitated and it was filtered to afford crude ODV base, which was further suspended in methanol (56.5 mL). To this suspension, conc. hydrochloric acid (35-37% w/v, 13.5 mL) was added to dissolve the solid, followed by addition of 25% w/v aqueous ammonia (15.5 mL) to precipitate the ODV as an off-white solid. The structure of the compound was confirmed on the basis of ¹H-NMR. Wt of the product=2.5 g; w/w yield=26%; purity (HLPC) in excess of 99.5%.

1. A process for the preparation of O-desmethylvenlafaxine (ODV, II), or a pharmaceutically acceptable salt thereof, comprising the reaction of venlafaxine, or a salt thereof, with a thiol reagent.

2. A process according to claim 1, wherein the thiol reagent is a dithiol.

3. A process according to claim 1 or 2, wherein the thiol reagent is selected from an alkyl, alkenyl, alkynyl, aryl, arylalkyl, arylalkenyl, arylalkynyl, alkylaryl, alkenylaryl or alkynylaryl thiol, each of which may optionally be substituted.

4. A process according to any one of claims 1 to 3, wherein the thiol reagent is selected from an optionally substituted alkyl, aryl, arylalkyl or alkylaryl thiol or a thiol prepared in-situ from an unsubstituted or substituted episulfide having alkyl, aryl, arylalkyl or alkylaryl substituents.

5. A process according to claim 4, wherein the thiol reagent is a straight-chained or branched alkyl or arylalkyl thiol reagent.

6. A process according to any one of claims 1 to 5, wherein the thiol reagent does not contain an aromatic group.

7. A process according to any one of claims 1 to 6, wherein the thiol reagent contains 1 to 20 carbon atoms.

8. A process according to any one of claims 1 to 7, wherein the thiol reagent is an aliphatic dithiol containing 1 to 20 carbon atoms.

9. A process according to claim 8, wherein the aliphatic dithiol is 1,2-ethane dithiol.

10. A process according to any one of claims 1 to 8, wherein the thiol reagent is an aminothiolate anion or an aminothiol.

11. A process according to claim 10, wherein the aminothiol or aminothiolate anion contains 1 to 20 carbon atoms.

12. A process according to claim 10 or 11, wherein the amino group of the aminothiol or aminothiolate anion is unsubstituted or substituted with one or more optionally substituted alkyl, alkenyl, alkynyl, aryl, arylalkyl, arylalkenyl, arylalkynyl, alkylaryl, alkenylaryl or alkynylaryl groups or mixtures thereof.

13. A process according to claim 12, wherein the amino group of the aminothiol or aminothiolate anion is either unsubstituted or substituted with one or more alkyl, aryl or arylalkyl groups or mixtures thereof, such as a N,N-dialkylaminoalkane thiol.

14. A process according to claim 13, wherein the N,N-dialkylaminoalkane thiol is 2-diethylaminoethane thiol.

15. A process according to claim 1 or 2, wherein the thiol reagent is an inorganic thiol.

16. A process according to claim 15, wherein the inorganic thiol is sodium thiol.

17. A process according to any one of the preceding claims, wherein the reaction solvent is selected from an alcohol, ethylene glycol, an ether of ethylene glycol or a mixture thereof.

18. A process according to claim 17, wherein the reaction solvent is selected from polyethylene glycol (e.g. polyethylene glycol 400), cellosolve or 1-butanol.

19. A process according to any one of the preceding claims, wherein the reaction solvent has a boiling point of at least 100° C.

20. A process according to any one of the preceding claims, wherein a thiolate anion is generated by treatment of the thiol reagent with a base, such as an alkoxide, preferably potassium t-butoxide, in the reaction solvent.

21. A process according to claim 20, wherein the alkoxide is not generated in-situ.

22. A process according to any one of the preceding claims, wherein the reaction is performed at a temperature within the range of 100° C. to 220° C.

23. A process according to claim 22, wherein the temperature is within the range of 120° C. to 150° C.

24. A process according to claim 23, wherein the temperature is within the range of 130° C. to 135° C.

25. A process according to any one of the preceding claims, wherein the venlafaxine or salt thereof is allowed to react with the thiol reagent for between 6 and 36 hours.

26. A process according to claim 25, wherein the venlafaxine or salt thereof is allowed to react with the thiol reagent for between 24 and 28 hours.

27. A process according to any one of the preceding claims, wherein during the work up procedure, the product is washed with a hydrocarbon solvent or a halogenated hydrocarbon solvent to remove process impurities.

28. A process according to claim 27, wherein the hydrocarbon solvent is selected from cyclohexane, toluene, xylene or mixtures thereof.

29. A process according to claim 27, wherein the halogenated hydrocarbon solvent is selected from dichloromethane, ethylene dichloride or mixtures thereof.

30. A process according to any one of the preceding claims, wherein the pharmaceutically acceptable salt of ODV prepared is selected from the succinate or fumarate salt.

31. A process according to any one of the preceding claims, wherein the salt of venlafaxine used is the hydrochloride salt.

32. A process according to any one of the preceding claims, wherein the crude ODV base formed is purified by mixing with an alcohol to form a suspension and adding acid followed by base.

33. A process for purifying ODV base, said process comprising the steps of mixing crude ODV base with an alcohol to form a suspension and adding acid followed by base.

34. A process according to claim 32 or 33, wherein the alcohol is selected from methanol, ethanol or isopropanol or a mixture thereof.

35. A process according to claim 34, wherein the alcohol is methanol.

36. A process according to any one of claims 32 to 35, wherein the acid used is an inorganic acid.

37. A process according to claim 36, wherein the inorganic acid is hydrochloric acid or sulfuric acid.

38. A process according to claim 37, wherein the inorganic acid is hydrochloric acid.

39. A process according to any one of claims 32 to 38, wherein the base used is an organic base.

40. A process according to claim 39, wherein the organic base is triethylamine or trimethylamine.

41. A process according to any one of claims 32 to 38, wherein the base used is an inorganic base.

42. A process according to claim 41, wherein the inorganic base is ammonia, sodium carbonate, potassium carbonate or sodium hydroxide.

43. A process according to any one of the preceding claims, wherein the ODV or pharmaceutically acceptable salt thereof obtained has a purity of 95% or more (as measured by HPLC).

44. A process according to any one of the preceding claims, wherein the ODV or pharmaceutically acceptable salt thereof is obtained in a yield of 25% or more.

45. A process according to any one of the preceding claims, wherein the process is carried out on an industrial scale.

46. ODV or a pharmaceutically acceptable salt thereof prepared by a process according to any one of the preceding claims.

47. ODV succinate prepared by a process according to any one of claims 1 to 45.

48. ODV fumarate prepared by a process according to any one of claims 1 to 45.

49. ODV or a pharmaceutically acceptable salt thereof according to any one of claims 46 to 48, for use in medicine.

50. ODV or a pharmaceutically acceptable salt thereof according to claim 49, for treating or preventing depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.

51. A pharmaceutical composition comprising ODV or a pharmaceutically acceptable salt thereof prepared by a process according to any one of claims 1 to 45.

52. A pharmaceutical composition according to claim 51, comprising ODV succinate.

53. A pharmaceutical composition according to claim 51, comprising ODV fumarate.

54. Use of a pharmaceutical composition according to any one of claims 51 to 53 for the preparation of a medicament for the treatment or prevention of depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease.

55. A method of treating or preventing depression, anxiety, a panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's disease, the method comprising administering to a patient in need thereof a therapeutically or prophylactically effective amount of ODV or a pharmaceutically acceptable salt thereof according to any one of claims 46 to 50.

56. A method according to claim 55, wherein the patient is a mammal.

57. A method according to claim 56, wherein the mammal is a human.
